(19) United States
(12) Patent Application Publication
(10) Pub. No.: US 2011/0297146 A1
(43) Pub. Date: Dec. 8, 2011

(54) LIQUID EJECTION HEAD TO BE USED FOR INHALER AND INHALER

(75) Inventors: Takatoshi Tanaka, Tokyo (JP); Satoshi Tsuchiya, Inagi-shi (JP)

(73) Assignee: CANON KABUSHIKI KAISHA, Tokyo (JP)

(21) Appl. No.: 13/146,041

(22) PCT Filed: Mar. 30, 2010
(86) PCT No.: PCT/JP2010/056122

§ 371 Date: Jul. 25, 2011

(30) Foreign Application Priority Data
Apr. 14, 2009 (JP) 2009-097790

(51) Int. Cl. A61M 11/00; B67D 7/06

(52) U.S. Cl. 128/200.14; 222/566

(57) ABSTRACT

A liquid ejection head has a plurality of liquid ejection ports 10 and a plurality of gas jet ports 11 that are arranged alternately. The liquid droplets ejected from the liquid ejection ports 10 are prevented from colliding with each other and can maintain a uniform particle size distribution as gas is jetted out from the gas jet ports 11 in a direction same as the direction in which liquid droplets are ejected from the liquid ejection ports 10.

### TECHNICAL FIELD

This invention relates to a liquid ejection head to be used for an inhaler designed to eject liquid droplets of a medicine or a pleasure intake item to allow a user to inhale them. The present invention also relates to an inhaler.

### BACKGROUND ART

Inkjet heads of inkjet recording apparatus are required not only to eject liquid droplets but also to control the direction of ejection of liquid droplets. To satisfy this requirement, methods have been proposed to control the direction of liquid droplets by means of one or more than one air flows.

For example, Japanese Patent Application Laid-Open No. H02-204049 discloses an inkjet head that prevents liquid droplets from deflecting so as to eject liquid droplets along a nearly straight trajectory by arranging air jet ports for driving air to be jetted out in a direction same as the direction of ink ejection at the outside of a plurality of ejection ports for ejecting ink. Japanese Patent Application Laid-Open No. 2007-301935 discloses an inkjet head showing an improved recording quality by arranging an air jet port substantially laid on an ink ejection port for ejecting ink so as to eject liquid droplets and air together and reduce tails of liquid droplets.

### DISCLOSURE OF THE INVENTION

Liquid ejection heads to be used for inhalers are required to produce an increased number of liquid droplets if compared with inkjet heads to be used for inkjet recording apparatus. However, the problems as described below arise when a liquid ejection head designed to produce an increased number of ejected liquid droplets is used for an inhaler.

The invention of Japanese Patent Application Laid-Open No. H02-204049 of arranging air jet ports at the outside of a plurality of liquid ejection ports cannot prevent liquid droplets from colliding with one another. Then, as a result, the particle size distribution of liquid droplets can be degraded. On the other hand, when an air jet port is arranged so as to be substantially laid on a liquid ejection port as in an inkjet head disclosed in Japanese Patent Application Laid-Open No. 2007-301935, residual liquid is left between a liquid ejection port and an air jet port so that the liquid ejection port is blocked to prevent liquid from being ejected.

Therefore, the object of the present invention is to provide a liquid ejection head to be used for an inhaler with few liquid droplets colliding with one another or adhering to the surface of the ejection head that can eject a group of micro liquid droplets showing a uniform particle size distribution.

According to the present invention, the above object is achieved by providing a liquid ejection head to be used for an inhaler, the liquid ejection head including; a plurality of liquid ejection ports for ejecting liquid to be inhaled by a user, and a means for generating energy for ejecting liquid from the plurality of liquid ejection ports, characterized in that gas jet ports for jetting out gas in a direction same as the direction of ejection of liquid droplets ejected from the liquid ejection ports are provided such that each of them is arranged between adjacently located liquid ejection ports.

Thus, according to the present invention, mutual collisions of liquid droplets ejected from the plurality of liquid ejection ports can be suppressed.

Then, as a result, if the number of ejected liquid droplets is increased, micro liquid droplets can be ejected to show a uniform particle size distribution.

Further features of the present invention will become apparent from the following description of exemplary embodiments with reference to the attached drawings.

### BRIEF DESCRIPTION OF THE DRAWINGS

FIG. 1A is a schematic perspective view of a liquid ejection head according to an embodiment of the present invention and FIG. 1B is an exploded schematic perspective view thereof.

FIG. 2A is a schematic perspective view of the liquid ejection section of the liquid ejection head illustrated in FIGS. 1A and 1B and FIG. 2B is an exploded schematic perspective view thereof.

FIG. 3 is a schematic perspective view of the ejection port plate of the liquid ejection head illustrated in FIGS. 1A and 1B.

FIG. 4A is a schematic plan view of part of the ejection ports of Example 1 and FIG. 4B is a schematic cross-sectional view taken along line 4B-4B in FIG. 4A.

FIG. 5 is a schematic plan view of the liquid ejection head of Example 1.

FIG. 6 is a schematic plan view of the liquid ejection head of Example 2.

FIG. 7 is a schematic plan view of the liquid ejection head of a modified example of Example 2.

FIG. 8A is a schematic plan view of part of the ejection ports of Example 3 and FIG. 8B is a schematic cross-sectional view taken along line 8B-8B in FIG. 8A.

FIG. 9 is a schematic plan view of the liquid ejection head of Example 3.

FIG. 10 is a schematic plan view of the liquid ejection head of a modified example of Example 3.

FIG. 11A is a schematic perspective view of an inhaler formed by using a liquid ejection head according to the present invention and FIG. 11B is a schematic elevation thereof in a state where its access cover is opened.

### BEST MODE FOR CARRYING OUT THE INVENTION

A liquid ejection head to be used for an inhaler according to the present invention is linked to a medicine dispenser and the liquid to be ejected may be selected from protein preparations including insulin, human growth hormone and gonadotropic hormone, nicotine and anesthetics.

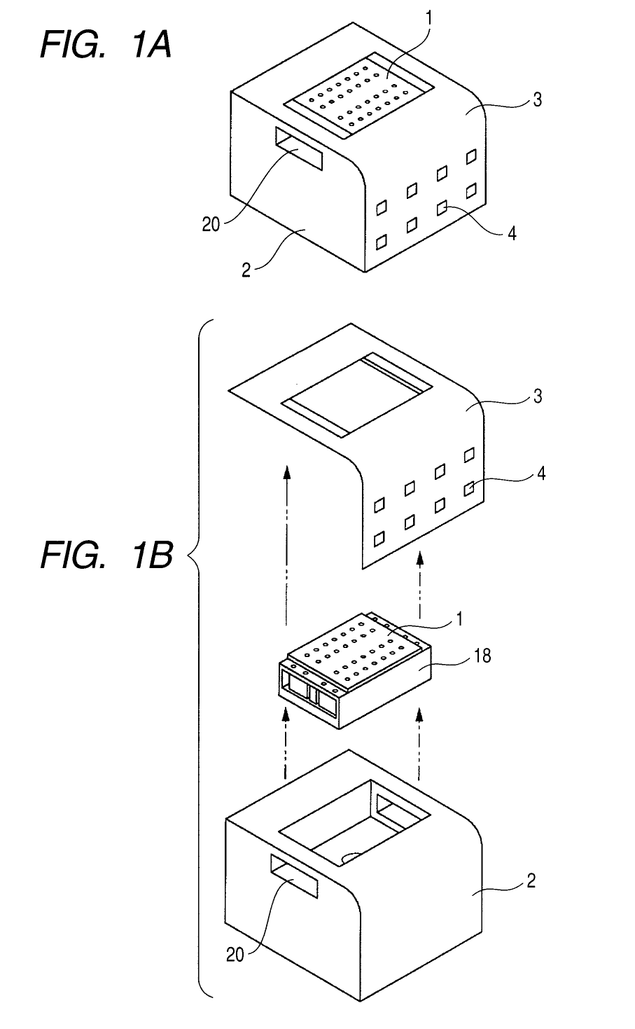

FIGS. 1A, 1B, 2A, 2B and 3 schematically illustrate a liquid ejection head to be used for an inhaler according to an embodiment of the present invention. The liquid ejection head is of a side shooter type designed to eject liquid droplets in a direction perpendicular to heating elements that generate thermal energy for causing film boiling to take place relative to liquid. The liquid ejection head includes a liquid ejection section 1, a chip holder 2 and an electric wiring tape 3.

The chip holder 2 is typically formed by resin molding and has a connection port for leading liquid from a liquid tank (not illustrated) to liquid supply ports 12 of a heating element substrate 18. The chip holder 2 has a function of holding a removable liquid tank (not illustrated) as part of its functions.

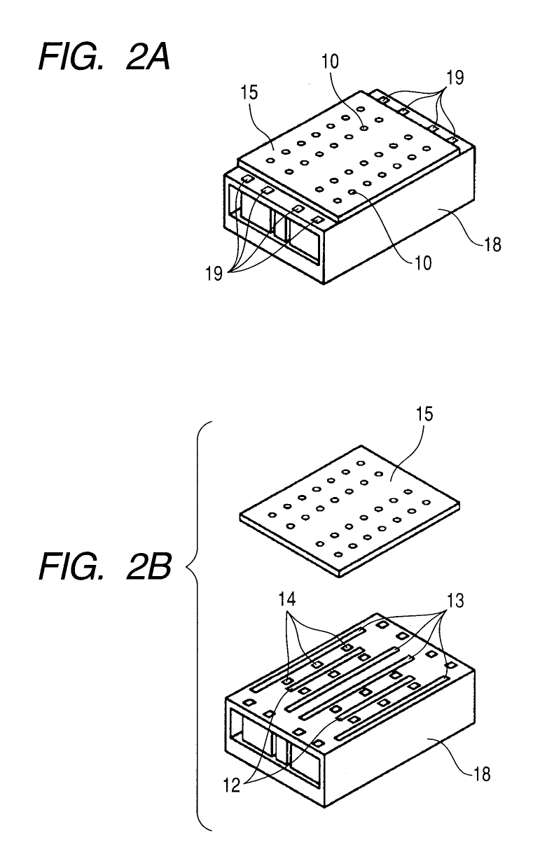

As illustrated in FIGS. 2A and 2B, the liquid supply ports 12 and gas supply ports 13 are long slot type through holes formed as liquid paths by anisotropic etching or laser processing utilizing the crystal orientation of Si in the heating element substrate 18 that is a 0.5 to 1 mm thick Si substrate. Each liquid supply port 12 is sandwiched by two rows of heating elements 14. The heating elements 14 and electric wiring of Au or the like (not illustrated) for supplying electric power to the heating elements 14 are formed by means of a film forming technique.

Furthermore, electrode sections 19 for supplying electric power to the electric wiring (not illustrated) are arranged at the opposite ends of the rows of the heating elements 14. The electric wiring is to apply electric signals for ejecting liquid to the heating element substrate 18. The electric wiring connects the electrode terminals that correspond to the electrode sections 19 of the heating element substrate 18 and external connection terminals 4 for receiving electric signals from the main-body of the inhaler.

The liquid supplied from the liquid supply ports 12 is then ejected from the liquid ejection ports 10 arranged respectively vis-à-vis the heating elements 14 by the air bubbles generated by the heating elements 14.

Gas is pressurized typically by means of a pressure pump (not illustrated) and introduced into the liquid ejection head from gas introduction ports 20. Then, the gas is jetted out from gas jet ports 11, each of which is arranged between two adjacent liquid ejection ports 10 by way of the gas supply ports 13. More specifically, a gas jet port 11 is arranged between two adjacent liquid ejection ports 10 in order to jet out gas in the direction same as the direction in which the liquid droplets ejected from the liquid ejection ports 10 are driven to proceed. The expression of “the same direction” does not necessarily means the direction that is rigorously parallel to the direction in which liquid droplets are ejected but a direction that is substantially same as the direction of ejection of liquid droplets. In this embodiment, it is a direction substantially perpendicular to the plane of the ejection port plate 15.

The heating elements 14 as means for generating energy necessary for ejecting liquid are arranged on the Si substrate at positions corresponding to the liquid paths. While the heating elements 14, liquid paths 16 and gas paths 17 are illustrated only by two or three in FIG. 2B, a plurality of liquid supply ports 12, gas supply ports 13 and heating elements 14 are actually arranged on a single Si substrate.

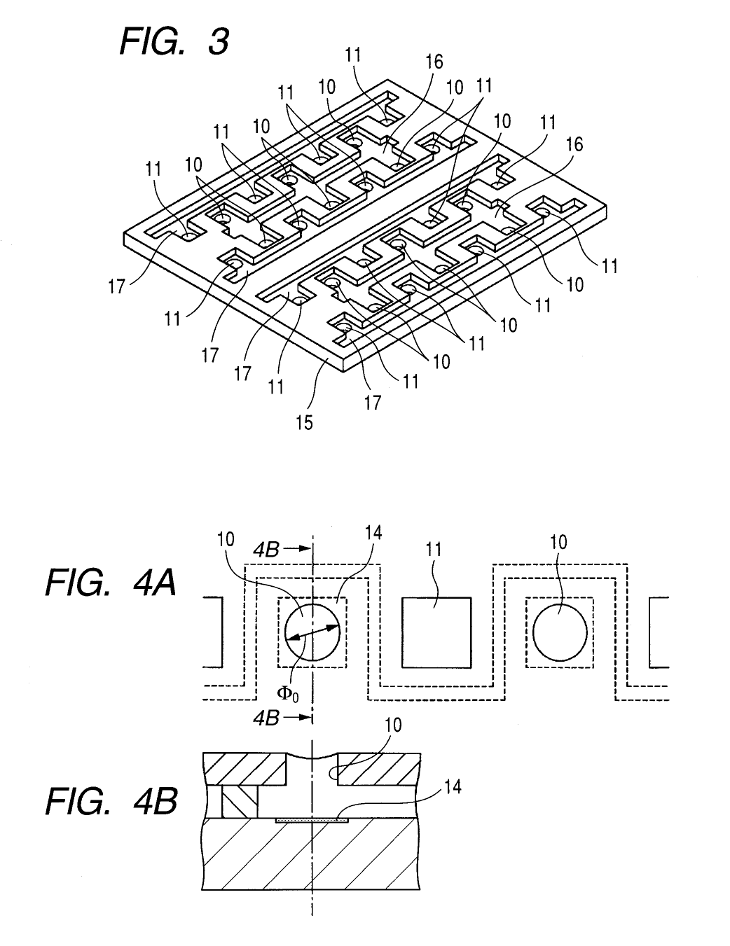

FIG. 3 is a bottom view of a typical structure of an ejection port plate 15. Path walls for forming liquid paths 16 that communicate with the liquid ejection ports 10, which correspond to the heating elements 14, and gas paths 17 that communicate with the gas jet ports 11 when mounted on the heating element substrate 18 are formed by means of a resin material and a photolithography technique. The liquid paths 16 and the gas paths 17 are surrounded by path walls and the heating element substrate 18 and heating elements 14 are arranged along each liquid path 16 at positions corresponding to the liquid ejection ports 10. Note that the means for generating energy for ejecting liquid are not limited to heating elements 14 that operate like heaters and oscillation energy generating elements such as piezoelectric elements may alternatively be employed.

### EXAMPLE 1

Liquid ejection ports 10 and gas jet ports 11 are arranged alternately in the liquid ejection head of Example 1 as illustrated in FIGS. 4A and 4B. Mutual collisions of liquid droplets can be suppressed as gas is jetted out from the gas jet ports 11 in a direction same as the direction of ejection of liquid droplets.

Additionally, liquid supply ports 12 and gas supply ports 13 are arranged alternately with the liquid ejection ports 10 and the gas jet ports 11 interposed between them.

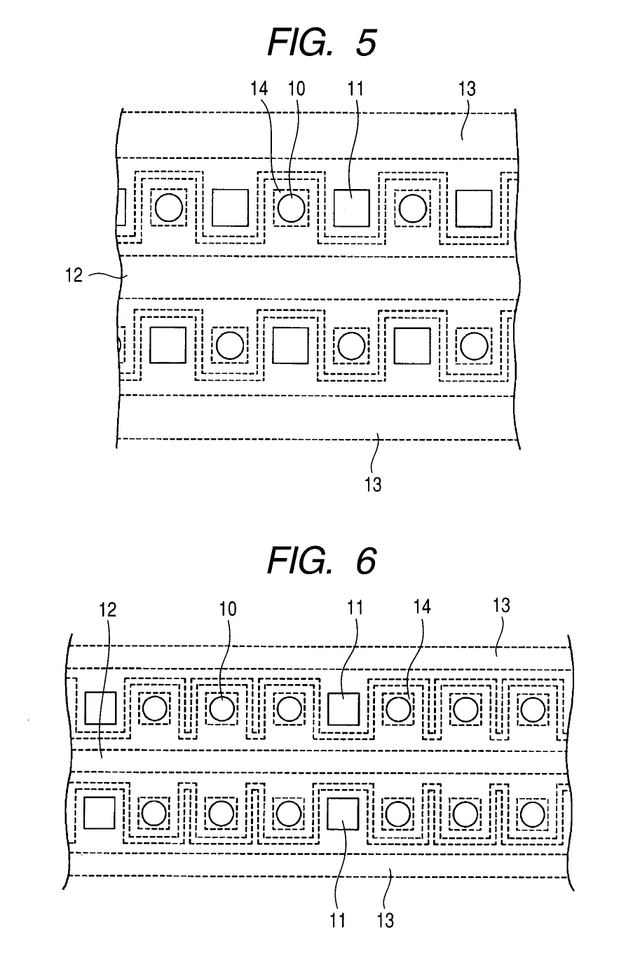

Alternatively, the liquid ejection ports 10 and the gas jet ports 11 may be arranged at offset positions as illustrated in FIG. 5. In other words, a gas jet port 11 is arranged at a position squarely opposite to a liquid ejection port 10 in a row of ejection ports with a liquid supply port 12 interposed between them. With such an arrangement, mutual collisions of liquid droplets ejected from the liquid ejection ports 10 arranged at opposite sides of a liquid supply port 12 can be successfully suppressed. Mutual collisions of liquid droplets can be suppressed when the gas flow rate of gas flows jetted out from the outermost groups of gas jet ports 11 is maximized out of the overall gas jet ports 11 in the liquid ejection section 1.

The gas jet ports 11 may be so arranged as to operate also as collection ports for collecting the residual liquid on the head surface before and after completion of ejecting liquid droplets. The residual liquid adhering to the head surface can be collected with air to prevent the residual liquid from blocking the liquid ejection ports 10 by reducing the gas pressure source to negative pressure.

### EXAMPLE 2

While all the liquid ejection ports 10 and the gas jet ports 11 are arranged alternately in Example 1, a plurality of liquid ejection ports 10 are arranged between two adjacent gas jet ports 11 in Example 2 as illustrated in FIG. 6. With such an arrangement, a large number of liquid droplets can be secured while suppressing degradation of particle size distribution due to mutual collisions of liquid droplets.

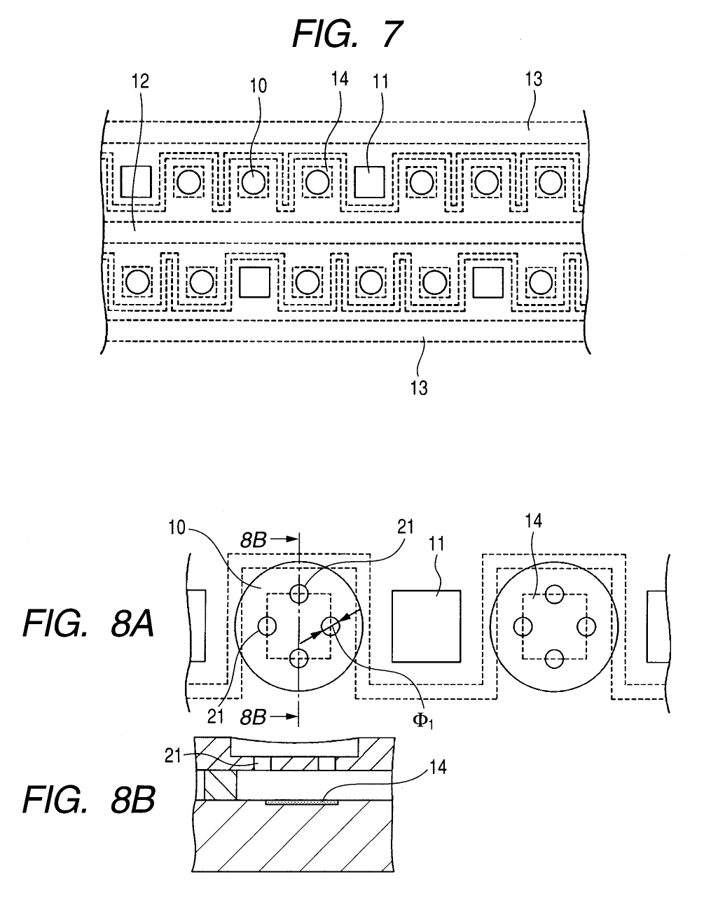

Alternatively, the liquid ejection ports 10 in each ejection port row and the gas jet ports 11 that are arranged oppositely with a liquid supply port 12 interposed between them may be arranged at offset positions as illustrated in FIG. 7. With such an arrangement, mutual collisions of liquid droplets ejected from the liquid ejection ports 10 located at opposite sides of a liquid supply port 12 can be suppressed.

The number of liquid ejection ports 10 arranged between two adjacent gas jet ports 11 is determined according to the permissible particle size distribution of liquid droplets. For example, assume that the permissible particle size distribution is 3±2 μm. In this case, suppose that liquid droplets of 3 μm are stably ejected from the liquid ejection ports 10. Then, the above-described requirement of particle size distribution is satisfied if four liquid droplets are combined into one but not if five or more liquid droplets are put together. In other words, the number of liquid ejection ports 10 are arranged preferably so as to satisfy the requirement of the formula shown below:

n≦(φₘₐₓ/φ₀)³ (formula 1),

where n is the number of successive liquid ejection ports 10, φ₀ is the particle size of liquid droplets ejected from the liquid ejection ports 10 and φₘₐₓ is the permissible upper limit value of the particle size.

Mutual collisions of liquid droplets can be suppressed when the gas flow rate of gas flows jetted out from the outermost groups of gas jet ports is maximized out of the overall gas jet ports 11 in the liquid ejection section 1.

Preferably, the gas jetted out from the gas jet ports 11 are controlled in terms of the content of gas produced when liquid is gasified so that liquid droplets may not be gasified easily. If liquid droplets are water droplets, preferably, the humidity rate of gas is controlled so as not to reduce the particle size of liquid droplets as a result of gasification of the liquid droplets. The gas that is jetted out preferably shows a humidity rate of not less than 80% and more preferably a humidity rate of not less than 90%.

The gas jet ports 11 may be so arranged as to operate also as collection ports for collecting the residual liquid on the head surface before and after completion of ejecting liquid droplets. The residual liquid adhering to the head surface can be collected with air to prevent the residual liquid from blocking the liquid ejection ports 10 by reducing the gas pressure source to negative pressure.

### EXAMPLE 3

In Example 3, as illustrated in FIGS. 8A and 8B, a plurality of through-holes of liquid ejection port 21 are arranged so as to make the recessed part of the liquid ejection port 10 communicate with a liquid path and a meniscus is formed in the recessed part to allow the through-holes of liquid ejection port 21 to be dipped in liquid. With such an arrangement, a greater number of liquid droplets can be produced in this example if compared with Example 1.

Examples of specific dimensions of a liquid ejection head of this example will be listed below. Each heating element 14 is a 10 μm square and the diameter of each liquid ejection port 10 is 18 μm while the diameter of each through-hole of liquid ejection port 21 is 3 μm. The thickness, by which each through-hole of liquid ejection port 21 extends is 1 μm. Additionally, the thickness of the ejection port plate 15 is 5 μm and the height of the path walls 16 is 5 μm.

The liquid ejection head has such a dimensional relationship that the length of each through-hole of liquid ejection port 21 the height of the path walls. In the liquid ejection head of this example, the thickness of the ejection port plate=5 μm and the height of the path walls=5 μm. Thus, the requirement of the relationship of the length of each through-hole of liquid ejection port≦the height of the path walls is met.

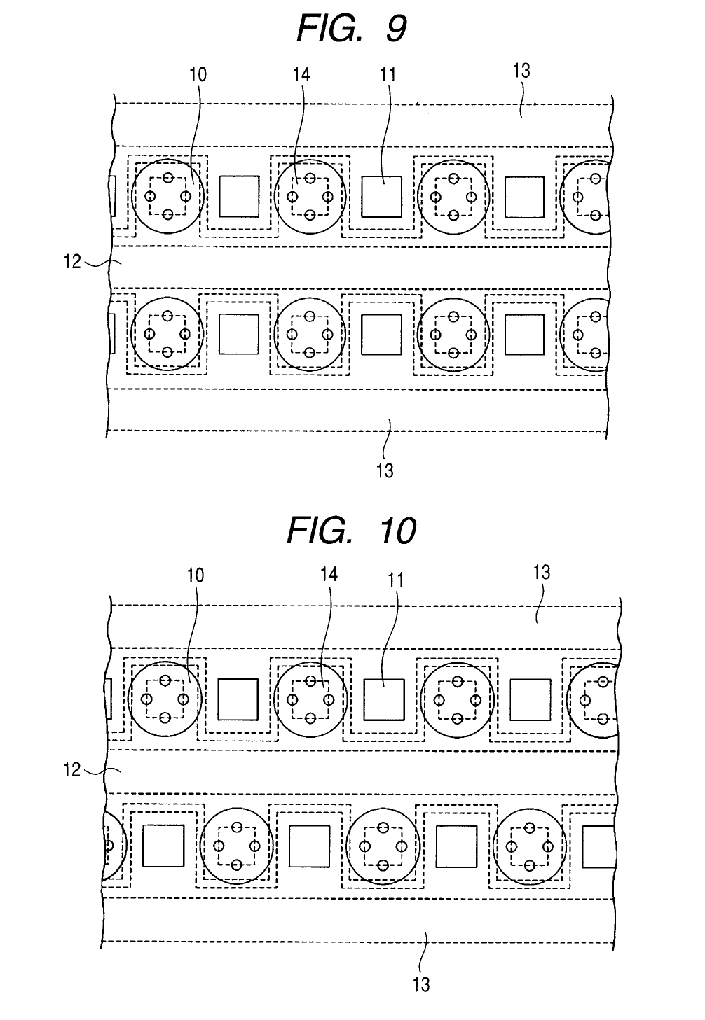

As illustrated in FIG. 9, the liquid supply ports 12 and the gas supply ports 13 are arranged in parallel with each other and alternately with liquid ejection ports 10 and gas jet ports 11 interposed between them.

The number of through-holes of liquid ejection port 21 is determined according to the permissible particle size distribution of liquid droplets. Assume here, for example, the permissible particle size distribution is 3±2 μm. In this case, suppose that liquid droplets of 3 μm are stably ejected from liquid ejection ports 10. Then, the above-described requirement of particle size distribution is satisfied if four liquid droplets are combined into one but not if five or more liquid droplets are put together. In other words, the number of through-holes 21 are arranged preferably so as to satisfy the requirement of the formula shown below:

n≦(φₘₐₓ/φₙ)³ (formula 2),

where n is the number of through-holes of liquid ejection port 21 arranged between two gas jet ports, φₙ is the diameter of each through-hole 21 of liquid ejection port 21 and φₘₐₓ is the permissible upper limit value of the size of liquid droplets.

Alternatively, the liquid ejection ports 10 in each ejection row and the gas jet ports 11 that are arranged oppositely with a liquid supply port 12 interposed between them may be arranged at offset positions as illustrated in FIG. 10. With such an arrangement, mutual collisions of liquid droplets ejected from the liquid ejection ports 10 located at opposite sides of a liquid supply port 12 can be suppressed. Mutual collisions of liquid droplets can be suppressed when the gas flow rate of gas flows jetted out from the outermost groups of gas jet ports 11 is maximized in the liquid ejection section 1.

The gas jet ports 11 may be so arranged as to operate also as collection ports for collecting the residual liquid on the head surface before and after completion of ejecting liquid droplets. The residual liquid adhering to the head surface can be collected with air to prevent the residual liquid from blocking the liquid ejection ports 10 by reducing the gas pressure source to negative pressure.

### EXAMPLE 4

An inhaler formed by using a liquid ejection head according to the present invention will be shown in Example 4. In the case of inhalation of a medicine such as insulin, it is known that the efficiency of absorption by blood is high and appropriate when the particle size of liquid droplets formed from the medicine is about 3 μm. The efficiency of absorption by blood is low and can result in a waste of medicine when the particle size is far from the above appropriate value. Mutual collisions of liquid droplets are reduced to by turn reduce the wasted medicine by utilizing a liquid ejection head according to the present invention.

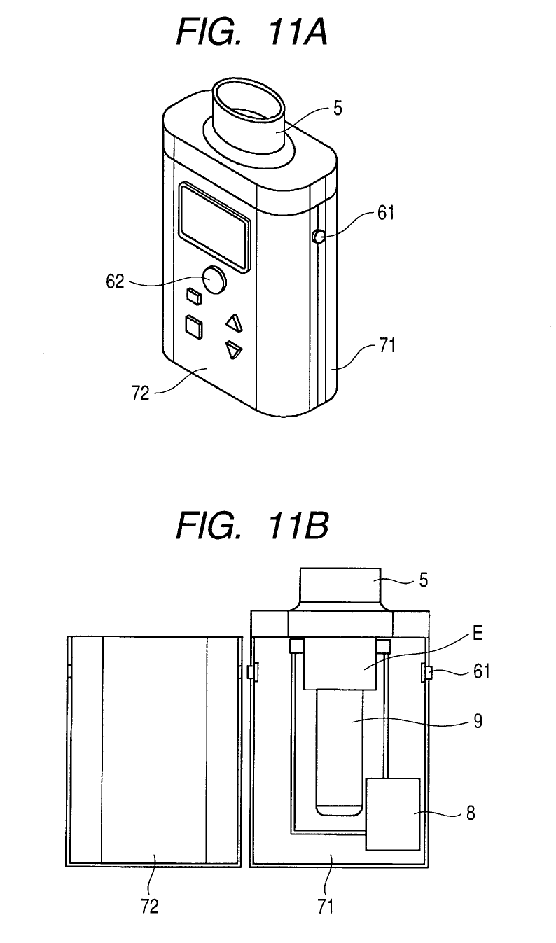

As illustrated in FIGS. 11A and 11B, a main-body cover 71 and an access cover 72 form an outer block of a medicine inhaler. The access cover 72 can be opened by unlocking the cover 72 by means of a lock release button 61. A mount part where a liquid ejection head as illustrated in FIGS. 1A and 1B is mounted is arranged in the inside of the main-body cover 71. The liquid ejection head can be electrically connected to a drive control section at the mount part of the liquid ejection head.

A liquid tank 9 is connected to the liquid ejection head E and a medicine such as insulin is contained in the liquid tank 9. The medicine is ejected as liquid droplets from the liquid ejection head E into an air flow duct communicating with a mouthpiece 5 so that the user can inhale liquid droplets by way of the mouthpiece 5.

The flow of operation of the medicine inhaler of this example for medicine inhalation will be described below. The user holds the mouthpiece 5 by lips and depresses an ejection switch 62 while inhaling. Air is introduced into the gas jet ports 11 by means of a pressurizing pump 8 contained in the medicine inhaler main-body or by an inhaling action on the part of the user. As described in detail in Examples 1 through 3, the gas jet ports 11 are arranged such that each of them is found between two adjacent liquid ejection ports 10. As air gets to the gas jet ports 11, it starts to be jetted out toward the air flow duct communicating with the mouthpiece 5. Immediately after air starts to be jetted out, the medicine is ejected as liquid droplets into the air flow duct communicating with the mouthpiece 5 from the liquid ejection ports 10 by the drive control section. The user inhales from the mouthpiece 5 the liquid droplets ejected into the liquid paths. As a required amount of medicine is ejected, the drive circuit stops operating and transmits a signal for notifying the user of the end of inhalation. The user ends the inhalation, recognizing the signal of the end of inhalation. The flow of operation for inhalation ends within 1 to 2 seconds in any case.

The above-described medicine inhaler of this example can eject a group of micro liquid droplets showing a uniform particle size distribution with few liquid droplets combined together or adhering to the surface of the ejection head so that the user can inhale a medicine with little waste of medicine.

The present invention is not limited to the above embodiments and various changes and modifications can be made within the spirit and scope of the present invention. Therefore, to apprise the public of the scope of the present invention, the following claims are made.

This application claims the benefit of Japanese Patent Application No. 2009-097790, filed on Apr. 14, 2009, which is hereby incorporated by reference herein in its entirety.

1. A liquid ejection head to be used for an inhaler, the liquid ejection head comprising:
a plurality of liquid ejection ports for ejecting liquid droplets to be inhaled by a user;
a means for generating energy for ejecting liquid from said plurality of liquid ejection ports; and
gas jet ports for jetting out gas in a direction of ejection of liquid droplets ejected from the liquid ejection ports, each of said gas jet ports being arranged between two adjacently located ones of said liquid ejection ports.

2. The liquid ejection head according to claim 1, wherein said liquid ejection ports and said gas jet ports are arranged alternately.

3. The liquid ejection head according to claim 1, further comprising liquid paths, wherein said liquid ejection ports communicate with said liquid paths by way of a plurality of through-holes of said liquid ejection ports and a meniscus is formed in each of said liquid ejection ports to allow said through-holes of said liquid ejection ports to be dipped in liquid.

4. The liquid ejection head according to claim 1, wherein said gas jet ports also operate as collection ports for collecting residual liquid on a surface of said head.

5. An inhaler for ejecting liquid and causing a user to inhale the liquid, comprising:
an air flow duct for guiding liquid droplets to be inhaled by the user into a mouthpiece with inspiration; and
the liquid ejection head for ejecting liquid droplets into said liquid paths according to claim 1.
